(19) United States
(12) Patent Application Publication
(10) Pub. No.: US 2009/0203888 A1
(43) Pub. Date: Aug. 13, 2009

(54) Isolated Photoprotein Aqdecay, and Its Use

(76) Inventors: Stefen Golz, Essen (DE); Eugene Vysotski, Krasnoyarsk (RU); Svetlana Markova, Krasnoyarsk (RU); Galina A. Stepanyuk, Krasnoyarsk region (RU); Ludmila Frank, Krasnoyarsk (RU)

Correspondence Address: Barbara A. Shimei;Director, Patents & Licensing, Bayer HealthCare LLC - Pharmaceuticals, 555 White Plains Road, Third Floor, Tarrytown, NY, 10591, US

(21) Appl. No.: 11/920,386

(22) PCT Filed: May 3, 2006
(86) PCT No.: PCT/EP2006/004116

§ 371 Date: Feb. 20, 2009

(51) Int. Cl. C07K 1/00; C07H 21/04; C07K 14/00; C12N 15/63; C12P 21/06

(52) U.S. Cl. 530/402; 536/23.1; 530/350; 435/320.1; 435/69.1

(57) ABSTRACT

The invention relates to the photoprotein AQdecay, to its nucleotide and amino acid sequences and to the activity and use of the photoprotein AQdecay.

The invention relates to the photoprotein AQdecay, to its nucleotide and amino acid sequences and to the activity and use of the photoprotein AQdecay.

### Photoproteins

The phenomenon of the generation of light by living organisms is designated bioluminescence. It is the result of biochemical reactions in cells, in which reactions the chemical energy is emitted in the form of light quanta (what is termed cold emission by means of chemoluminescence). While the light which is produced in this way is monochromatic, since it is emitted in connection with a discrete electron transfer, it can be displaced by secondary luminescent dyes (e.g. fluorescent proteins in the case of luminescent jellyfish of the genus Aequoria) into spectral regions of longer wavelength.

Bioluminescence has a diversity of biological functions: at an ocean depth of between 200 and 1000 m (mesopelagial), about 90% of all living organisms luminesce. In this case, the luminescent signals are employed for attracting partners, for deception and as a lure. Glowworms and fireflies also use the light signals for seeking partners. On the other hand, the significance of the luminescence of bacteria, fungi and single-cell algae is unclear. It is assumed that it is used for coordinating many single individuals in a large population or else represents a type of biological clock.

A large number of coelenterates are bioluminescent (Morin et al., 1974). These organisms emit blue or green light. As an isolated protein, aequorin, which is derived from Aequoria victoria(Shimomura et al., 1969) and which, in 1962, was the first light-producing protein to be identified, emitted a blue light, and not a green light as observed phenotypically in the case of Aequoria victoria. The green fluorescent protein (GFP) which, as a result of being activated by aequorin, causes Aequoria victoria to appear phenotypically green was subsequently isolated from this medusa (Johnson et al., 1962; Hastings et al., 1969; Inouye et al., 1994). Other photoproteins which have also been identified and described are clytin (Inouye et al., 1993), mitrocomin (Fagan et al., 1993) and obelin (Illarionov et al., 1995).

TABLE 1
Overview of some photoproteins. The table gives the name,
the organism from which the protein has been isolated and the
identification number (Acc. No.) of the database entry.

| Name | Organism | Identification No. |
|---|---|---|
| Obelin | Obelia geniculata | AAL86372 |
| Clytin | Clytia gregaria | CAA49754 |
| Aequorin | Aequorea macrodactyla | AAK02061 |
| Aequorin | Aequorea parva | AAK02060 |
| Mitrocomin | Mitrocoma cellularia | AAA29298 |
| Pholasin | Pholas dactylus | AAM18085 |
| ? | Symplectoteuthis oualaniensis | AX305029 |

TABLE 2
Overview of some photoproteins. The table gives the organism
from which the protein has been isolated, the name of the photoprotein
and a selection of patents or applications.

| Organism | Fluorescent protein | Patent/Application |
|---|---|---|
| Obelia geniculata | Obelin | WO03006497 |
| Clytia gregaria | Clytin | WO03006497 |
| Aequoria victoria | Aequorin | WO200168824 |
| | | U.S. Pat. No. 0,908,909 |
| | | U.S. Pat. No. 6,152,358 |
| | | JP-0176125 |
| Pholas dactylus | Pholasin | WO0028025 |
| | | GB-0024357 |

Bioluminescence is nowadays used in technology in a wide variety of ways, e.g. in the form of bioindicators of environmental pollution or in biochemistry for sensitively detecting proteins or for quantifying particular compounds, or as what are termed reporters in connection with investigating gene regulation in the cell.

The photoproteins differ not only in their nucleotide and amino acid sequences but also in their biochemical and physical properties.

It has been demonstrated that the physical and biochemical properties of photoproteins can be altered by altering the amino acid sequences of these proteins. Examples of mutagenized photoproteins are described in the literature (U.S. Pat. No. 6,495,355; U.S. Pat. No. 5,541,309; U.S. Pat. No. 5,093,240; Shimomura et al., 1986).

The abovementioned photoproteins generate light by oxidizing coelenterazine (Haddock et al., 2001; Jones et al., 1999).

### Reporter Systems

In general, genes whose gene products can be readily detected using simple biochemical or histochemical methods are termed reporter genes or indicator genes. At least 2 types of reporter gene are distinguished.
1. Resistance genes. This is the term used for genes whose expression confers, on a cell, resistance to antibiotics or other substances whose presence in the growth medium leads to the death of the cell if the resistance gene is absent.
2. Reporter genes. The products of reporter genes are used in genetic manipulation as fused or unfused indicators. The commonest reporter genes include beta-galactosidase (Alam et al., 1990), alkaline phosphatase (Yang et al., 1997; Cullen et al., 1992), and luciferases and other photoproteins (Shinomura, 1985; Phillips G N, 1997; Snowdowne et al., 1984).

The emission of photons in the visible spectral range, with this emission being effected by means of excited emitter molecules, is termed luminescence. In contrast to fluorescence, the energy is not, in this case, supplied from the exterior in the form of radiation of shorter wavelength.

A distinction is made between chemiluminescence and bioluminescence. A chemical reaction which leads to an excited molecule which itself luminescences when the excited electrons return to the basal state is termed chemiluminescence. If this reaction is catalysed by an enzyme, the phenomenon is then referred to as being bioluminescence. The enzymes involved in the reaction are generally termed luciferases.

### Preparing the Mutant

In order to prepare the mutant, molecular biological methods were used to insert the mutations at position 89 [Y89F] (GenBank #AAA27716; position 89 of SEQ ID 5) and position 139 [Y139F] (GenBank #AAA27716; position 139 of SEQ ID 5). Stratagene's “Quick change” method (catalogue number #200521; revision #063001b; 2003 edition) was used for this purpose. (SEQ ID NO: 3) and (SEQ ID NO: 4) were used as primers. The vector was designated pET22b-AQdecay.

### AQdecay

Photoproteins which exhibited altered spectral or biochemical properties as a result of individual amino acids having been substituted have already been described in the literature. These photoproteins include obelin W92F (Vysotski et al., 2003) and aequorin (Shrestha et al., 2002; Ohmiya et al., 1993).

The aequorin mutant AQdecay exhibits a release of light which is chronologically altered as compared with the photoprotein aequorin or other photoproteins.

The mutation at position 139, which is responsible for the chronological change in the light release, was combined with a mutation at position 89. The change at position 89 has already been described and leads to a change in the spectral properties of the photoprotein. In addition to exhibiting the chronological change in light release, the selected combination also exhibits altered spectral properties. It is possible to combine the change at position 139 with substitutions of other amino acids. It is also possible to combine the change at position 139 with the wild-type sequence of the remaining aequorin photoprotein.

The photoprotein AQdecay surprisingly exhibits a retarded light-release or luminescence kinetics which has not previously been described. In addition to the possible uses which are customary, this property enables the photoprotein to be used specifically for investigating reactions or mechanisms involving very rapid calcium release in eukaryotic cells or other systems. The kinetics of the release of light from previously described photoprotein mutants or wild-type photoproteins is described as “flash” kinetics since, following activation (e.g. with calcium), the light is released over a very short period of time and the reaction then comes to a standstill or at least becomes markedly weaker. Special measuring instruments are required for measuring this rapid kinetics. The described photoprotein mutant AQdecay, or its equivalents, not only make it possible to use other measuring instruments or measuring methods but also, in particular, make it possible to investigate very rapid kinetics. This kinetics can arise, for example, in connection with ion channels belonging to the P2X family.

The spectrum of aequorin, having a maximum at 470 nm, has been described (Shimomuro et al., 1966). The spectral properties of coelenterazines have been surveyed by Shinomuro (Shimomura et al., 2000).

With an identity of 99%, the photoprotein AQdecay exhibits the highest degree of homology at the amino acid level with aequorin from Aequoria victoria (shown in example 8). The BLAST method was used for comparing the sequences (Altschul et al., 1997).

The invention relates to the photoprotein AQdecay, which has the amino acid sequence which is represented by SEQ ID NO: 2. The invention likewise relates to the nucleic acid molecule depicted in SEQ ID NO: 1.

The invention also relates to functional equivalents of AQdecay. Functional equivalents are those proteins which possess comparable physicochemical properties.

The invention relates to aequorin photoproteins which, in the region of amino acid positions 129-149, 124-134, preferably 137-141, in particular 138-140 (based on GenBank #AAA27716), exhibit one or more amino acid mutations which lead to the bioluminescence properties being changed. In addition, the invention relates to aequorin photoproteins which, at position 139 (based on GenBank #AAA27716), exhibit an amino acid mutation which leads to a change in the bioluminescence properties. In this connection, aequorin photoproteins can also be photoproteins which exhibit, in the region of amino acids 134-145, a motif which is similar to that of the truncated aequorin (GenBank #AAA27716). In this context, regions having a similar motif are regarded as being sequences which, in this region, exhibit an identity of 80%, preferably of 90%.

The invention relates to combinations, with mutations in the amino acid position 139 region, of aequorin photoproteins which, in the region of amino acid positions 79-99, 84-94, preferably 87-91, in particular 88-90 (based on GenBank #AAA27716), exhibit one or more amino acid mutations which lead to a change in the fluorescence spectrum or bioluminescence spectrum. In addition, the invention relates to combinations, with mutations in the amino acid position 139 region, of aequorin photoproteins which exhibit, in position 89 (based on GenBank #AAA27716), an amino acid mutation which leads to a change in the fluorescence spectrum or bioluminescence spectrum. In this connection, preference is given to those photoproteins which exhibit a maximum in the fluorescence spectrum or bioluminescence spectrum in the range of 480-520 nm, preferably of 485-515 nm, particularly preferably in the range of from 490-510 nm, 495 to 505, or, in particular, at 500 nm. In this connection, aequorin photoproteins can also be those photoproteins which, in the region of amino acids 84-94, exhibit a motif which is similar to that of the truncated aequorin (GenBank #AAA27716). In this connection, regions having a similar motif are regarded as being those sequences which, in this region, exhibit an identity of 80%, preferably of 90%.

Functional fragments of the AQdecay protein, or nucleic acids encoding such fragments, are likewise in accordance with the invention.

Truncated functional fragments of other proteins according to the invention, or nucleic acids which encode these fragments, likewise form part of the invention.

The photoprotein AQdecay is suitable for being used as a reporter gene for cellular systems, especially for receptors, for ion channels, for transporters, for transcription factors or for inducible systems.

The photoprotein AQdecay is also suitable for being used as a reporter gene for labelling, identifying and characterizing cell organelles, especially for mitochondria.

The photoprotein AQdecay is also suitable for being used as a reporter gene for determining parameters within and outside cell organelles, especially mitochondria, especially calcium concentrations.

The photoprotein AQdecay is suitable for being used as a reporter gene in bacterial and eukaryotic systems, especially in mammalian cells, in bacteria, in yeasts, in bacculoviruses and in plants.

The photoprotein AQdecay is suitable for being used as a reporter gene for cellular systems in combination with bioluminescent or chemiluminescent systems, especially systems using luciferases, using oxygenases or using phosphatases.

The photoprotein AQdecay is suitable for being used as a fusion protein, especially for receptors, for ion channels, for transporters, for transcription factors, for proteinases, for kinases, for phosphodiesterases, for hydrolases, for peptidases, for transferases, for membrane proteins and for glycoproteins.

The photoprotein AQdecay is suitable for being used for immobilization, especially by antibodies, by biotin, or by magnetic or magnetizable supports.

The photoprotein AQdecay is suitable for being used as a protein for energy transfer systems, especially FRET (fluorescence resonance energy transfer), BRET (bioluminescence resonance energy transfer), FET (field effect transistors), FP (fluorescence polarization) and HTRF (homogeneous time-resolved fluorescence) systems.

The photoprotein AQdecay is suitable for labelling substrates or ligands, especially for proteases, for kinases or for transferases.

The photoprotein AQdecay is suitable for being expressed in bacterial systems, especially for titre determination, or as a substrate for biochemical systems, especially for proteinases and kinases.

The photoprotein AQdecay is suitable for being used as a label, especially coupled to antibodies, coupled to enzymes, coupled to receptors, or coupled to ion channels and other proteins.

The photoprotein AQdecay is suitable for being used as a reporter gene in connection with searching for pharmacological active compounds, especially in HTS (high throughput screening).

The photoprotein AQdecay is suitable for being used as a reporter gene in connection with characterizing, identifying and investigating ion channels, especially of the p2x, TRP, SCN, KCN, CNG or ACCN type.

The photoprotein AQdecay is suitable for being used as a component of detection systems, especially for ELISA (enzyme-linked immunosorbent assay), for immunohistochemistry, for Western blotting or for confocal microscopy.

The photoprotein AQdecay is suitable for being used as a label for analysing interactions, especially for protein-protein interactions, for DNA-protein interactions, for DNA-RNA interactions, for RNA-RNA interactions or for RNA-protein interactions (DNA: deoxyribonucleic acid; RNA: ribonucleic acid).

The photoprotein AQdecay is suitable for being used as a label or fusion protein for expression in transgenic organisms, especially in mice, in rats, in hamsters and other mammals, in primates, in fish, in worms or in plants.

The photoprotein AQdecay is suitable for being used as a label or fusion protein for analysing embryonic development.

The photoprotein AQdecay is suitable for being used as a label by way of a coupling mediator, especially by way of biotin, by way of NHS(N-hydroxysulphosuccinimide) or by way of CN—Br.

The photoprotein AQdecay is suitable for being used as a reporter coupled to nucleic acids, especially to DNA or to RNA.

The photoprotein AQdecay is suitable for being used as a reporter coupled to proteins or peptides.

The photoprotein AQdecay is suitable for being used as a reporter for measuring intracellular or extracellular calcium concentrations.

The photoprotein AQdecay is suitable for characterizing signal cascades in cellular systems.

The photoprotein AQdecay which is coupled to nucleic acids or peptides is suitable for being used as a probe, especially for Northern blots, for Southern blots, for Western blots, for ELISA, for nucleic acid sequencing, for protein analyses or for chip analyses.

The photoprotein AQdecay is suitable for labelling pharmacological formulations, especially infectious agents, antibodies or “small molecules”.

The photoprotein AQdecay is suitable for being used for geological investigations, especially for ocean, groundwater and river currents.

The photoprotein AQdecay is suitable for being expressed in expression systems, especially in in-vitro translation systems, in bacterial systems, in yeast systems, in baculovirus systems, in viral systems or in eukaryotic systems.

The photoprotein AQdecay is suitable for visualizing tissues or cells in connection with surgical interventions, especially in connection with invasive interventions, in connection with noninvasive interventions and in connection with minimally invasive interventions.

The photoprotein AQdecay is also suitable for labelling tumour tissues and other phenotypically altered tissues, especially in connection with histological investigation or in connection with surgical interventions.

The invention also relates to the purification of the photoprotein AQdecay, especially as a wild-type protein, as a fusion protein or as a mutagenized protein.

The photoprotein AQdecay is suitable for simultaneously measuring different reporter genes in an expression system (multiplexing).

The invention also relates to the use of the photoprotein AQdecay in the field of cosmetics, especially of bath additives, of lotions, of soaps, of body dyes, of toothpaste and of body powders.

The invention also relates to the use of the photoprotein AQdecay for dyeing, in particular, foodstuffs, bath additives, ink, textiles and plastics.

The invention also relates to the use of the photoprotein AQdecay for dyeing paper, especially greetings cards, paper products, wallpapers and handicraft articles.

The invention also relates to the use of the photoprotein AQdecay for dyeing liquids, especially for water pistols, for fountains, for beverages and for ice.

The invention also relates to the use of the photoprotein AQdecay for producing toys, especially finger paint and makeup.

The invention relates to nucleic acid molecules which encode the polypeptide disclosed by SEQ ID NO: 2 or functional equivalents or functional fragments thereof.

The invention furthermore relates to nucleic acid molecules or functional equivalents or functional fragments thereof which are selected from the group consisting of
a) nucleic acid molecules which encode a polypeptide which comprises the amino acid sequence disclosed by SEQ ID NO: 2;
b) nucleic acid molecules which contain the sequence depicted by SEQ ID NO: 1;
c) nucleic acid molecules whose complementary strands hybridize, under stringent conditions, with a nucleic acid molecule from a) or b) and whose expression products exhibit the biological function of a photoprotein;
   a stringent hybridization of nucleic acid molecules is carried out, at 68° C., in an aqueous solution which contains 0.2×SSC (1×standard saline-citrate=150 mM NaCl, 15 mM trisodium citrate) (Sambrook et al., 1989).
d) nucleic acid molecules which differ from those mentioned under c) due to the degeneracy of the genetic code.

The invention relates to the abovementioned nucleic acid molecules in which the sequence contains a functional promoter 5′ to the photoprotein-encoding sequence or to the sequence encoding the leader sequence or signal sequence.

The invention also relates to nucleic acid molecules as previously described which form part of recombinant DNA or RNA vectors.

The invention relates to organisms which harbor such a vector.

The invention relates to photoproteins which are encoded by the previously described nucleotide sequences.

The invention relates to methods for expressing the photoprotein polypeptides according to the invention in bacteria, eukaryotic cells or in-vitro expression systems.

The invention also relates to methods for purifying/isolating a photoprotein polypeptide according to the invention.

The invention relates to the use of the photoprotein-encoding nucleic acids according to the invention as marker genes or reporter genes, in particular for searching for pharmacological active compounds and for diagnostics.

The invention relates to the use of the photoproteins according to the invention or a photoprotein-encoding nucleic acid according to the invention as labels or reporters and, respectively, as marker gene or reporter gene.

The invention relates to the use of the photoprotein AQdecay (SEQ ID NO: 2), or of its functional fragments or equivalents, or to the use of a photoprotein AQdecay-encoding nucleic acid, or of its functional fragments or equivalents, as label or reporter and, respectively, as marker or reporter gene, in particular for searching for pharmacological active compounds and for diagnostics.

The invention relates to the use of the nucleic acid depicted in SEQ ID NO: 1 as a marker gene or reporter gene, in particular for searching for pharmacological active compounds and for diagnostics.

The invention also relates to polyclonal or monoclonal antibodies which recognize a polypeptide according to the invention.

The invention also relates to monoclonal or polyclonal antibodies which recognize the photoprotein AQdecay (SEQ ID NO:2).

The invention also relates to a nucleic acid, as described in the previous paragraphs, which contains a functional promoter 5′ to the coding sequence.

The invention encompasses recombinant DNA or RNA vectors which contain the previously described nucleic acids.

Organisms which harbor a vector as previously described are likewise in accordance with the invention.

A polypeptide which is encoded by a nucleic acid sequence as described above likewise forms part of the invention.

A method for expressing the previously mentioned polypeptides in bacteria, eukaryotic cells or in-vitro expression systems is also in accordance with the invention.

A method for purifying/isolating a polypeptide according to the invention likewise forms part of the invention.

The invention relates to the use of a nucleic acid according to the invention as a marker gene or reporter gene.

The invention also relates to the use of a photoprotein according to the invention as a label or reporter.

The use of a polypeptide according to the invention in combination with one or more luciferases and/or one or more photoproteins also forms part of the invention.

A photoprotein, or a functional fragment thereof, which possesses one or more mutations in the 129-149, 124-134, preferably 137-141, in particular 138-140, region (based on GenBank #AAA27716), and which exhibits an altered, especially retarded, bioluminescence signal, is in accordance with the invention.

A nucleic acid molecule which comprises a sequence which encodes a protein in accordance with the two previous paragraphs is likewise in accordance with the invention.

The invention furthermore relates to a method for preparing a photoprotein, characterized in that one or more mutations are introduced into a photoprotein in the region defined by positions 129-149, 124-134, preferably 137-141, in particular 138-140, based on GenBank #AAA27716, with this resulting in a change in the bioluminescence.

A photoprotein which is prepared by a method as described in the previous paragraph is likewise in accordance with the invention.

The invention also relates to other photoproteins which, as a result of one or more changes in the amino acid sequence, exhibit an altered light-release kinetics.

The invention also relates to the use of other altered photoproteins for the described uses of the photoprotein AQdecay.

Photoproteins having an altered light-release kinetics, in particular a retarded light release or prolonged period in which light is released, are particularly suitable for being used as reporter genes in cell-based methods, especially in searching for and characterizing pharmacological active compounds and especially in diagnostics.

Photoproteins having an altered light-release kinetics, in particular a retarded light release or prolonged period in which light is released, are particularly suitable for investigating ion channels.

The invention also relates to codon-optimized variants of the proteins according to the invention for altering the biochemical or physicochemical properties, especially improved expression, especially altered stability.

The invention also relates to fusions of the proteins according to the invention with recognition peptides for the purpose of transporting or locating the proteins according to the invention into/in cell organelles or compartments.

The invention also relates to variants of the proteins according to the invention which lead to a change in the spectral properties, in the luminescence intensity, in the substrate specificity, in the use of cofactors, in the calcium affinity or in other physicochemical or biochemical properties.

### Expressing the Photoproteins of the Invention

The production of a molecule which, after the gene has been introduced into a suitable host cell, enables the foreign gene which is cloned into an expression vector to be transcribed and translated is termed expression. Expression vectors contain the control signals which are required for expressing genes in prokaryotic or eukaryotic cells.

In principle, expression vectors can be constructed in two different ways. In the case of what are termed transcription fusions, the protein encoded by the cloned-in foreign gene is synthesized as an authentic, biologically active protein. For this purpose, the expression vector carries all the 5′ and 3′ control signals which are required for the expression.

In the case of what are termed translation fusions, the protein encoded by the cloned-in foreign gene is expressed, together with another protein which can be detected readily, as a hybrid protein. The 5′ and 3′ control signals which are required for the expression, including the start codon and, possibly, a part of the sequences encoding the N-terminal regions of the hybrid protein to be formed, originate from the vector. The additional protein moiety which is inserted not only in many cases stabilizes the protein, which is encoded by the cloned-in foreign gene, against breakdown by cellular proteases; it can also be used for detecting and isolating the hybrid protein which is formed. The expression can take place either transiently or stably. Suitable host organisms are bacteria, yeasts, viruses or eukaryotic systems.

### Purifying the Photoproteins of the Invention

The isolation of proteins (after they have been overexpressed as well) is frequently termed protein purification. A large number of established methods are available for purifying proteins.

The solid/liquid separation is a basic operation in connection with isolating proteins. This procedural step is required when separating cells from the culture medium, when clarifying the crude extract after having disrupted the cells and removing the cell debris, and when separating off sediments after precipitations, etc. It takes place by means of centrifugation and filtration.

In order to obtain intracellular proteins, the cell wall must be destroyed or rendered permeable. High-pressure homogenizers or agitator ball mills or glass bead mills are used for this purpose, depending on the scale and the organism. Mechanical cell disintegrators and ultrasonic treatment are used, inter alia, on the laboratory scale.

Both in the case of extracellular proteins and in the case of intracellular proteins (following cell disruption), various precipitation methods using salts (in particular ammonium sulphate) or organic solvents (alcohols or acetone) represent rapid and efficient methods for concentrating proteins. When intracellular proteins are being purified, it is desirable to remove the soluble nucleic acids (precipitation with, for example, streptomycin sulphate or protamine sulphate). When extracellular proteins are being isolated, carriers (e.g. starch or kieselguhr) are frequently added before adding the precipitating agents in order to obtain sediments which are easier to handle.

Numerous chromatographic methods and partition methods (absorption chromatography and ion exchange chromatography, gel filtration, affinity chromatography and electrophoreses) are available for high-degree purification. Column chromatography is also used on an industrial scale. Affinity chromatography, which makes possible purification factors of up to several 100s per step, is especially important for the laboratory scale.

Extracellular proteins accrue in relatively dilute solutions. Just like extracellular proteins, they have to be concentrated before being subjected to further use. In addition to the methods which have already been mentioned, ultrafiltration has proved to be of value, on an industrial scale as well.

Inorganic salts which accompany proteins are frequently undesirable in the case of specific applications. They can be removed by, inter alia, gel filtration, dialysis and diafiltration.

A large number of proteins are used as dry preparations. Important drying methods are vacuum drying, freeze drying and spray drying.

### Nucleotide and Amino Acid Sequences

The photoprotein AQdecay is encoded by the following nucleotide sequence (SEQ ID NO: 1):

5′-
ATGTCAGTCAAGCTTACACCAGACTTCGACAACCCAAAATGGATTGGACG
ACACAAGCACATGTTTAATTTTCTTGATGTCAACCACAATGGAAGGATCT
CTCTTGACGAGATGGTCTACAAGGCGTCCGATATTGTTATAAACAATCTT
GGAGCAACACCTGAACAAGCCAAACGTCACAAAGATGCTGTAGAAGCCTT
CTTCGGAGGAGCTGGAATGAAATATGGTGTAGAAACTGAATGGCCTGAAT
TTATCGAAGGATGGAAAAGACTGGCTTCCGAGGAATTGAAAAGGTATTCA
AAAAACCAAATCACACTTATTCGTTTATGGGGTGATGCATTGTTCGATAT
CATTGACAAAGACCAAAATGGAGCTATTTCACTGGATGAATGGAAAGCAT
TCACCAAATCTGCTGGCATCATCCAATCGTCAGAAGATTGCGAGGAAACA
TTCAGAGTGTGCGATATTGATGAAAGTGGACAGCTCGATGTTGATGAGAT
GACAAGACAACATTTAGGATTTTGGTACACCATGGATCCTGCTTGCGAAA
AGCTCTACGGTGGAGCTGTCCCCTAA -3′.

This yields an amino acid sequence of (SEQ ID NO: 2):

MTSEQYSVKLTPDFDNPKWIGRHKHMFNFLDVNHNGRISLDEMVYKASDIVINNLGATPE
QAKRHKDAVEAFFGGAGMKYGVETEWPEFIEGWKRLASEELKRYSKNQITLIRLWGDAL
FDIIDKDQNGAISLDEWKAFTKSDGIIQSSEDCEETFRVCDIDESGQLDVDEMTRQHLGFW
YTMDPACEKLYGGAVP

### Primers:

(SEQ ID NO: 3):
5′- GAATGGCCTGAATTTATCGAAGGATGGAA -3′
(SEQ ID NO: 4):
5′- TTCCATCCTTCGATAAATTCAGGCCATTC -3′
(SEQ ID NO: 5):
5′- GAATGGAAAGCATTCACCAAATCTGCTG -3′
(SEQ ID NO: 6):
5′- CAGCAGATTTGGTGAATGCTTTCCATTC -3′

The photoprotein aequorin (Genbank: AAA27716) possesses the following amino acid sequence (SEQ ID NO: 7). Positions 89 and 139 are printed in bold and underlined.

MTSEQYSVKLTPDFDNPKWIGRHKHMFNFLDVNHNGRISLDEMVYKASDIVINNLGATPE
QAKRHKDAVEAFFGGAGMKYGVETEWPEYIEGWKRLASEELKRYSKNQITLIRLWGDAL
FDIIDKDQNGAISLDEWKAYTKSDGIIQSSEDCEETFRVCDIDESGQLDVDEMTRQHLGFW
YTMDPACEKLYGGAVP

These sequences are reproduced in the sequence listing.

### BRIEF DESCRIPTION OF THE FIGURES

FIG. 1 shows the plasmid map of the vector pET22b-AQdecay.

FIG. 2 shows the plasmid map of the vector pcDNA3-AQdecay.

FIG. 3 shows the result of the eukaryotic expression of AQdecay in CHO cells. The experiment took place as described in example 4. (Y=relative light units, RLU; X=log conc. of ATP/mol/l)

FIG. 4 shows the result of the bacterial expression of AQdecay. The experiment took place as described in example 3. (Y=relative light units, RLU; X=time in seconds; black curve: AQdecay; grey curve: wild-type aequorin)

FIG. 5 shows the bioluminescence kinetics of AQdecay (expression in CHO cells). The experiment took place as described in example 4. (Y=relative light units, RLU; X=time in seconds; black curve: AQdecay; grey curve: wild-type aequorin)

### EXAMPLES

### Example 1

In order to prepare the mutant, the mutations were inserted at position 132 (of the truncated aequorin; GenBank #AAA27716) using molecular biological methods. The “Quick change” method provided by Stratagene (USA) was used for this purpose. The primers employed were (SEQ ID NO: 3) and (SEQ ID NO: 4). The cDNA was inserted into the NdeI/XhoI cleavage site of the vector pET22b (Novagen). The vector was designated pET22b-AQdecay.

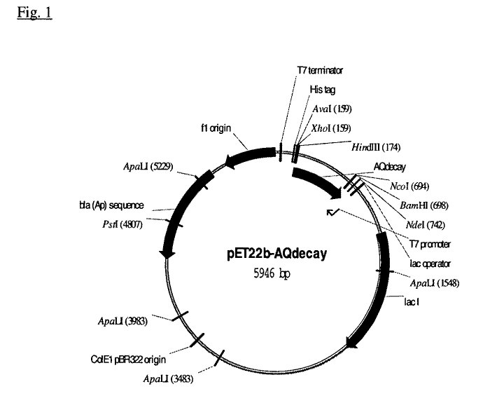

FIG. 1 shows the plasmid map of the vector pET22b-AQdecay.

### Example 2

The plasmid pcDNA3.1(+) supplied by Clontech was used as vector for preparing the construct which is described below. The derivative of the vector was designated pcDNA3-AQdecay. The vector pcDNA3-AQdecay was used for expressing AQdecay in eukaryotic systems.

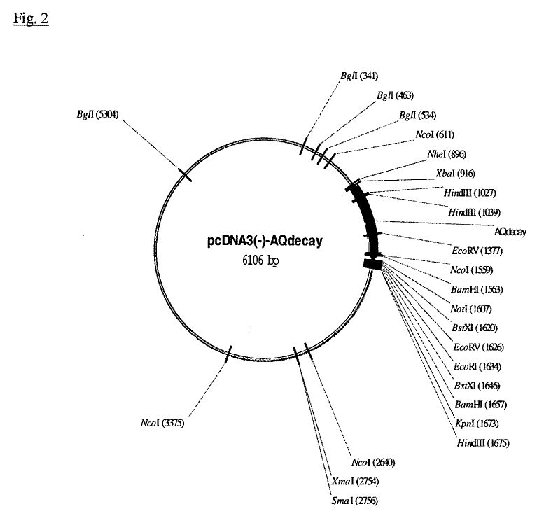

FIG. 2 shows the plasmid map of the vector pcDNA3-AQdecay.

### Example 3

### Bacterial Expression

Bacterial expression was effected in E. coli by transforming the bacteria with the expression plasmid pET22b-AQdecay. The transformed bacteria were incubated at 37° C. for 3 hours in LB medium and expression was induced in accordance with the manufacturer's (Novagen) instructions. The induced bacteria were harvested by centrifugation, resuspended in 50 mM Tris/HCl (pH 9.0)+5 mM EDTA and disrupted by means of ultrasonication. The lysate was then centrifuged for 15 minutes at 13 000 rpm (16 000 rcf) and the supernatant was taken off. The supernatant (1:5; 1:10; 1:20 and 1:50 dilutions with Tris/HCl pH 9.0) was incubated for 3 hours in the dark with coelenterazine (10E-07 M coelenterazine in Tris/HCl pH 9.0). The bioluminescence was measured in a luminometer directly after adding 5 mM calcium chloride. The measurement integration time was 40 seconds.

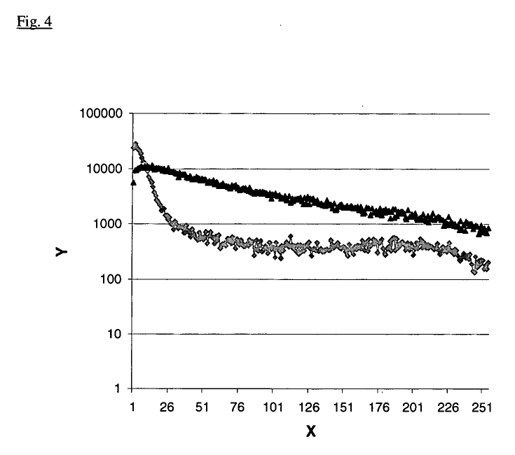

FIG. 4 shows the kinetics of the measurement of AQdecay bioluminescence in bacteria.

### Example 4

### Eukaryotic Expression

Constitutive eukaryotic expression took place on CHO cells as a result of transfecting the cells with the expression plasmids pcDNA3-AQdecay and pcDNA3.1(+) in transient experiments. For this, 10 000 cells were plated out, per well, in DMEM-F12 medium on 96-well microtitre plates and the plates were incubated overnight at 37° C. Transfection was effected using the Fugene 6 kit (Roche) in accordance with the manufacturer's instructions. The transfected cells were incubated overnight at 37° C. in DMEM-F12 medium. The medium was then removed and replaced with 50 μl of coelenterazine (10E-07 M coelenterazine in PBS). The cells were incubated at 28° C. for 24 hours, after which ATP (adenosine triphosphate) was added to a final concentration of 1 μM. The measurement in a luminometer was started directly after making this addition. The integration time was 1 second, with a total measurement duration of 60 seconds.

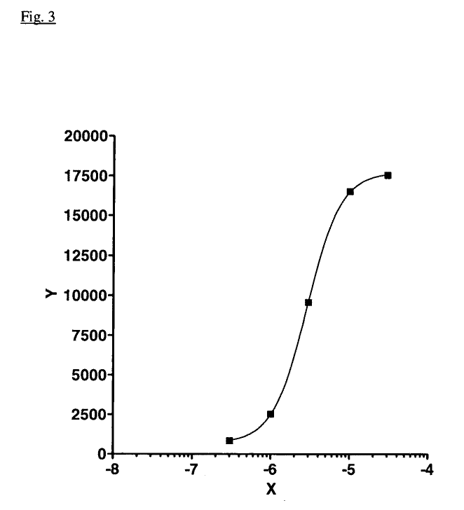

FIG. 3 shows the results of the measurement of AQdecay bioluminescence in CHO cells.

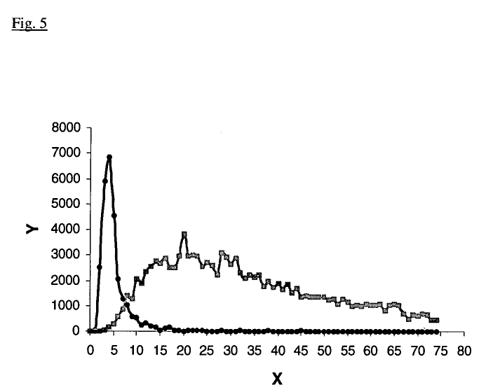

FIG. 5 shows the kinetics of the measurement of AQdecay bioluminescence in CHO cells.

### Example 5

### Blast

Result of a blast analysis of AQdecay at the amino acid level.

>emb|CAC93774.1| unnamed protein product [Aequorea victoria]
Length=196, Score=410 bits (1054), Expect=e-113, Identities=194/196 (98%), Positives=196/196 (100%)
>pir∥A26623 aequorin-1 precursor-hydromedusa (Aequorea victoria) sp|P07164|AEQ1_AEQVI Aequorin 1 precursor gb|AAA27716.11 aequorin 1 precursor
Length=196, Score=410 bits (1054), Expect=e-113, Identities=194/196 (98%), Positives=196/196 (100%)
>gb|AAB14842.1| Sequence 1 from U.S. Pat. No. 5,541,309 gb|AAA55424.1| Sequence 2 from Patent EP 0187519
Length=196, Score=407 bits (1046), Expect=e-113, Identities=193/196 (98%), Positives=195/196 (99%)
>gb|AAB14845.1| Sequence 4 from U.S. Pat. No. 5,541,309
Length=196, Score=405 bits (1041), Expect=e-112, Identities=192/196 (97%), Positives=194/196 (98%)
>gb|AAB14846.1| Sequence 5 from U.S. Pat. No. 5,541,309
Length=196, Score=405 bits (1040), Expect=e-112, Identities=192/196 (97%), Positives=194/196 (98%)
>gb|AAB14844.1| Sequence 3 from U.S. Pat. No. 5,541,309
Length=196, Score=405 bits (1040), Expect=e-112, Identities=192/196 (97%), Positives=194/196 (98%)>
emb|CAC93778.1| unnamed protein product [Aequorea victoria]
Length=196, Score=402 bits (1034), Expect=e-111, Identities=191/196 (97%), Positives=193/196 (98%)
>dbj|BAC81730.1| apoaequorin [Aequorea victoria]
Length=196, Score=401 bits (11031), Expect=e-111, Identities=189/196 (96%), Positives=195/196 (99%)
>emb|CAC93779.1| unnamed protein product [Aequorea victoria]
Length=196, Score=400 bits (1029), Expect=e-111, Identities=190/196 (96%), Positives=192/196 (97%)
>emb|CAC93780.1| unnamed protein product [Aequorea victoria]
Length=196, Score=400 bits (1028), Expect=e-110, Identities=190/196 (96%), Positives=192/196 (97%)
>pdb|1SL8|A Chain A, Calcium-Loaded Apo-Aequorin From Aequorea victoria
Length=191, Score=395 bits (1015), Expect=e-109, Identities=187/190 (98%), Positives=189/190 (99%)
>gb|AAB14843.1| Sequence 2 from U.S. Pat. No. 5,541,309
Length=189, Score=394 bits (1011), Expect=e-108, Identities=186/189 (98%), Positives=188/189 (99%)
>emb|CAC93777.1| unnamed protein product [Aequorea victoria]
Length=189, Score=391 bits (1005), Expect=e-108, Identities=185/189 (97%), Positives=187/189 (98%)
>emb|CAC93781.1| unnamed protein product [Aequorea victoria]
Length=189, Score=391 bits (1004), Expect=e-108, Identities=184/189 (97%), Positives=187/189 (98%)
>emb|CAC93775.1| unnamed protein product [Aequorea victoria]
Length=196, Score=384 bits (985), Expect=e-105, Identities=176/196 (89%), Positives=192/196 (97%)
>dbj|BAC81731.1| apoaequorin [Aequorea victoria]
Length=196, Score=384 bits (985), Expect=e-105, Identities=176/196 (89%), Positives=192/196 (97%)

### Example 6

### Blast

Result of a blast analysis of AQdecay at the nucleic acid level:

>gb|M16103.1|AEV AEQA A. victoria (jellyfish) aequorin 1 mRNA, complete cds
Length=672, Score=1104 bits (557), Expect=0.0, Identities=569/573 (99%)
>dbj|AB103337.1| Aequorea victoria mRNA for apoaequorin, clone:UTAEQ04
Length=591, Score=961 bits (485), Expect=0.0, Identities=551/573 (96%)
>dbj|AB103338.1| Aequorea victoria mRNA for apoaequorin, clone:UTAEQ09
Length=591, Score=739 bits (373), Expect=0.0, Identities=523/573 (91%)
>gb|L29571.1|AEV AQ440X Aequorea victoria aequorin (AQ440) mRNA, complete cds
Length=925, Score=731 bits (369), Expect=0.0, Identities=522/573 (91%)
>gb|M11394.1|AEV AEQD Aequorea victoria (jellyfish) aequorin mRNA, complete cds
Length=861, Score=731 bits (369), Expect=0.0, Identities=522/573 (91%)
>dbj|AB103336.1| Aequorea victoria mRNA for apoaequorin, clone:UTAEQ01
Length=591, Score=724 bits (365), Expect=0.0, Identities=521/573 (90%)
>dbj|AB103339.1| Aequorea victoria mRNA for apoaequorin, clone:UTAEQ11
Length=591, Score=716 bits (361), Expect=0.0, Identities=520/573 (90%)
>gb|AY601106.1| Aequorea victoria aequorin mRNA, complete cds
Length=600, Score=716 bits (361), Expect=0.0, Identities=517/569 (90%)
gb|AY604002.1| Aequorea victoria clone AEQ_V44A modified aequorin mRNA, complete cds
Length=600, Score=708 bits (357), Expect=0.0, Identities=516/569 (90%)
>gb|AY604001.1| Aequorea victoria clone AEQ_Q168R modified aequorin mRNA, complete cds
Length=600, Score=708 bits (357), Expect=0.0, Identities=516/569 (90%)
>gb|AY604000.1| Aequorea victoria clone AEQN26D modified aequorin mRNA, complete cds
Length=600, Score=708 bits (357), Expect=0.0, Identities=516/569 (90%)
>gb|AY603999.1| Aequorea victoria clone AEQ_L170I modified aequorin mRNA, complete cds
Length=600, Score=708 bits (357), Expect=0.0, Identities=516/569 (90%)
>gb|AY603998.1| Aequorea victoria clone AEQ_F149S modified aequorin mRNA, complete cds
Length=600, Score=708 bits (357), Expect=0.0, Identities=516/569 (90%)
>gb|AY603997.1| Aequorea victoria clone AEQ_E35G modified aequorin mRNA, complete cds
Length=600, Score=708 bits (357), Expect=0.0, Identities=516/569 (90%)
>gb|AY603996.1| Aequorea victoria clone AEQ_E128G modified aequorin mRNA, complete cds Length=600, Score=708 bits (357), Expect=0.0, Identities=516/569 (90%)
>gb|AY603995.1| Aequorea victoria clone AEQ_D153G modified aequorin mRNA, complete cds
Length=600, Score=708 bits (357), Expect=0.0, Identities=516/569 (90%)
>gb|AY603994.1| Aequorea victoria clone AEQ_D117G modified aequorin mRNA, complete cds
Length=600, Score=708 bits (357), Expect=0.0, Identities=516/569 (90%)
>gb|AY603993.1| Aequorea victoria clone AEQ-Q168A-L170V modified aequorin mRNA, complete cds
Length=600, Score=676 bits (341), Expect=0.0, Identities=512/569 (89%)

### Example 7

FIG. 7 shows the alignment of AQdecay with aequorin (wildtype; wt) at the amino acid level.

| | |
|---|---|
| WT | MTSEQYSVKLTPDFDNPKWIGRHKHMFNELDVNHNGRISLDEMVYKASDIVINNL |
| DECAY | MTSEQYSVKLTPDFDNPKWIGRHKHMFNFLDVNHNGRISLDEMVYKASDIVINNL |
| WT | GATPEQAKRHKDAVEAFFGGAGMKYGVETEWPEFIEGWKRLASEELKRYSKNQIT |
| DECAY | GATPEQAKRHKDAVEAFFGGAGMKYGVETEWPEYIEGWKRLASEELKRYSKNQIT |
| WT | LIRLWGDALFDIIDKDQNGAISLDEWKAFTKSDGIIQSSEDCEETFRVCDIDESG |
| DECAY | LIRLWGDALFDIIDKDQNGAISLDEWKAYTKSDGIIQSSEDCEETFRVCDIDESG |
| WT | QLDVDEMTRQHLGFWYTMDPACEKLYGGAVP |
| DECAY | QLDVDEMTRQHLGFWYTMDPACEKLYGGAVP |

### Example 8

### Kinetic Analysis of AQdecay Expressed in Bacteria

In order to analyse the bioluminescence of AQdecay kinetically, E. coli BL21 (DE3) was transformed with pET22b-AQdecay or pET22b (without any integrated cDNA). The bacteria were propagated and disrupted as described in example 3. The measurement data were collected for a period of 60 seconds using an integration time of 1 second.

FIG. 4 shows the results of the kinetic analysis of AQdecay in bacteria.

### Example 9

### Kinetic Analysis of AQdecay Expressed in CHO Cells

In order to analyse the bioluminescence of AQdecay kinetically, CHO (Chinese hamster ovarian cells) cells were transiently transfected with pcDNA3-AQdecay or pcDNA3 (without any integrated cDNA). The transfection and measurement were carried out as described in example 4. The measurement data were collected for a period of 60 seconds using an integration time of 1 second.

FIG. 5 shows the results of the kinetic analysis of AQdecay in CHO cells.

### Example 10

### Using AQdecay in Multiplexing Experiments

The photoprotein AQdecay is suitable for being used as a component in multiplexing readout methods in which several reporter genes (e.g. luciferases or photoproteins) are used in an experimental mixture. For this, AQdecay-expressing CHO cells were mixed, in a ratio of 1:1 (or 1:2, 1:3, . . . ) with CHO cells which were expressing the wild-type aequorin. The cells which were expressing the wild-type aequorin were additionally expressing a G protein-coupled receptor (e.g. neuromedin U receptor 2). The cell mixture was plated out on 96-well, 384-well or 1536-well microtitre plates, which were then incubated at 37° C. for 24 hours.

The cells were then loaded with coelenterazine (as described in example 4). Adding the G protein receptor agonist results in calcium being released intracellularly. This release can be read out using the wild-type aequorin (release of light by wild-type aequorin). The AQdecay of the second cell type can be activated by subsequently adding an agonist (e.g. ATP) which activates an endogenous CHO receptor.

### Example 11

### Locating AQdecay in Cell Organelles or Compartments

The photoprotein AQdecay, or its equivalents, is/are suitable for being fused with peptides, leader sequences, translocation signals, proteins or protein fragments for the purpose of transport into, or location in, special cell compartments or organelles. For the purpose of transporting, and subsequently locating, the photoprotein AQdecay, the photoprotein according to the invention was fused with the peptide MSVLTPLLLRGLTGSARRLPVPRAKIHSLPPEGKL. Fusion of the peptide upstream of the AQdecay amino acid sequence leads to the fusion protein being translocated into the mitochondria of the eukaryotic host cell. The mitochondrially located photoprotein AQdecay can be used for measuring the calcium concentration within the mitochondria. The fusion of the described peptide upstream of the amino acid sequence of the AQdecay photoprotein was effected at the nucleic acid level using standard molecular biological methods.

### REFERENCES/PATENTS

U.S. Pat. No. 6,495,355
U.S. Pat. No. 5,541,309
U.S. Pat. No. 5,093,240
US-0908909
U.S. Pat. No. 6,152,358
JP-0176125
GB-0024357
WO03006497
WO200168824
Alam J, Cook J L. Reporter genes: application to the study of mammalian gene transcription. Anal Biochem. 1990 August 1; 188(2):245-54
Altschul, Stephen F., Thomas L. Madden, Alejandro A. Schäffer, Jinghui Zhang, Zheng Zhang, Webb Miller, and David J. Lipman (1997); Gapped BLAST and PSI-BLAST: a new generation of protein database search programs; Nucleic Acids Res. 25:3389-3402
Chiesa A, Rapizzi E, Tosello V, Pinton P, de Virgilio M, Fogarty K E, Rizzuto R. Recombinant aequorin and green fluorescent protein as valuable tools in the study of cell signalling. Biochem J. 2001 Apr. 1; 355(Pt 1):1-12.
Claros, M. G., Vincens, P. (1996); Computational method to predict mitochondrially imported proteins and their targeting seqeunces. Eur. J. Biochem 241, 779-786.
Cullen Bryan R., Malim Michael H., Secreted placental alkaline phosphatase as a eukaryotic reporter gene. Methods in Enzymology. 216:362ff
Fagan T F, Ohmiya Y, Blinks J R, Inouye S, Tsuji F I. Cloning, expression and sequence analysis of cDNA for the Ca(2+)-binding photoprotein, mitrocomin. FEBS Lett. 1993 Nov. 1; 333(3):301-5
Hastings, J. W. and Morin, J. G. (1969) Comparative biochemistry of calcium-activated photoproteins from the ctenophore, Mnemiopsis and the coelenterates Aequorea, Obelia, and Pelagia. Biol. Bull. 137, 402.
Haddock S H, Rivers T J, Robison B H. Can coelenterates make coelenterazine? Dietary requirement for luciferin in cnidarian bioluminescence. Proc Natl Acad Sci USA 2001 Sep. 25; 98(20):11148-51
Inouye S, Tsuji F I. (1994) Aequorea green fluorescent protein. Expression of the gene and fluorescence characteristics of the recombinant protein. FEBS Lett 1994 March 21; 341(2-3):277-80
Inouye S, Tsuji F I. Cloning and sequence analysis of cDNA for the Ca(2+)-activated photoprotein, clytin. FEBS Lett. 1993 January 11; 315(3):343-6.
Illarionov B A, Bondar V S, Illarionova V A, Vysotski E S. Sequence of the cDNA encoding the Ca(2+)-activated photoprotein obelin from the hydroid polyp Obelia longissima. Gene. 1995 Feb. 14; 153(2):273-4.
Jones K, Hibbert F, Keenan M. Glowing jellyfish, luminescence and a molecule called coelenterazine. Trends Biotechnol 1999 December; 17(12):477-81
Johnson, F. H., Shimomura, O., Saiga, Y., Gershman, L. C., Reynolds, G. T., and Waters, J. R. (1962) Quantum efficiency of Cypridina luminescence, with a note on that of Aequorea. J. Cell. Comp. Physiol. 60, 85-103.
Morin, J. G. and Hastings, J. W. (1971) Biochemistry of the bioluminescence of colonial hydroids and other coelenterates. J. Cell. Physiol. 77, 305-311.
Ohmiya Y, Tsuji F I. Bioluminescence of the Ca(2+)-binding photoprotein, aequorin, after histidine modification. FEBS Lett. 1993 Apr. 12; 320(3):267-70.
Phillips G N. Structure and dynamics of green fluorescent protein. Curr Opin Struct Biol. 1997 December; 7(6):821-7
Sambrook, J., Fritsch, E. Maniatis, T. 1989, Molecular cloning. A laboratory manual Vol 1-3, Cold Spring Harbor, New York: Cold Spring Harbor Laboratory Press
Shimomura O, Johnson F H. Properties of the bioluminescent protein aequorin. Biochemistry. 1969 October; 8(10):3991-7
Shimomura O., Bioluminescence in the sea: photoprotein systems. Symp Soc Exp Biol. 1985; 39:351-72
Shimomura, O. and Teranishi K. (2000) Luminescence 15, 51-58.
Shimomura O. Isolation and properties of various molecular forms of aequorin. Biochem J. 1986 Mar. 1; 234(2):271-7.
Shimomura, O. and Johnson, F. H. (1966) in: Bioluminescence in Progress (Johnson, F. H. and Haneda, Y., Eds.) pp. 496-521, Princeton University Press, Princeton, N. J.
Shrestha S, Paeng I R, Deo S K, Daunert S. Cysteine-free mutant of aequorin as a photolabel in immunoassay development. Bioconjug Chem. 2002 March-April; 13(2):269-75
Snowdowne K W, Borle A B. Measurement of cytosolic free calcium in mammalian cells with aequorin. Am J. Physiol. 1984 November; 247(5 Pt 1):C396-408.
Vysotski E S, Liu Z J, Markova S V, Blinks J R, Deng L, Frank L A, Herko M, Malikova N P, Rose J P, Wang B C, Lee J. Violet bioluminescence and fast kinetics from W92F obelin: structure-based proposals for the bioluminescence triggering and the identification of the emitting species. Biochemistry. 2003 May 27; 42(20):6013-24.
Ward, W. W. (1998) Biochemical and physical properties of green fluorescent protein. In: Green Fluorescent Protein Properties, Applications, and Protocols (Chalfie, M. and Kain, S., eds) pp. 45-70. Wiley-Liss, Inc.
Yang Te-Tuan, Sinai Parisa, Kitts Paul A. Kain Seven R., Quantification of gene expression with a secreted alkaline phosphatase reporter system. Biotechnique. 1997 23(6) 1110ff

1. A nucleic acid molecule, or a functional fragment thereof, which is selected from the group consisting of:
a) a nucleic acid molecules which encodes a polypeptide which comprises the amino acid sequence disclosed by SEQ ID NO: 2;
b) a nucleic acid molecule which contains the sequence depicted by SEQ ID NO: 1;
c) a nucleic acid molecule whose complementary strands hybridize, under stringent conditions, with a nucleic acid molecule from a) or b) and whose expression products exhibit the biological function of a photoprotein; and
d) a nucleic acid molecules which differs from those mentioned under c) due to the degeneracy of the genetic code.

2. A polypeptide, or a functional fragment thereof, which is encoded by a nucleic acid sequence according to claim 1 and possesses the property of a photoprotein.

3. A photoprotein, or a functional fragment thereof, which possesses one or more mutations in positions 129 to 149 based on SEQ ID NO: 7 and which exhibits an altered chronological bioluminescence.

4. A photoprotein, or a functional fragment thereof, which possesses a mutation in position 139 based on SEQ ID NO: 7 and which exhibits an altered chronological bioluminescence.

5. A nucleic acid molecule which comprises a sequence which encodes a protein according to claims 3 and 4.

6. A nucleic acid molecule according to claim 1 or 5 which contains a functional promoter 5′ to the coding sequence.

7. A recombinant DNA or RNA vector which contains a nucleic acid molecule according to claim 6.

8. A host cell which harbors a vector according to claim 7.

9. An oligonucleotide having more than 10 consecutive nucleotides which are identical with, or complementary to, a constituent sequence of a nucleic acid molecule according to claim 1 or 5.

10. A method for expressing the polypeptides according to claims 2, 3 or 4 in a bacterial or eukaryotic cell or an in-vitro expression system.

11. The method according to claim 10 further comprising the step of isolating the polypeptide.

12. (canceled)

13. (canceled)

14. A method for preparing a photoprotein, comprising introducing one or more mutations in the region defined by positions 137 to 141 of SEQ ID NO: 7, thereby changing the chronological bioluminescence.

15. A photoprotein, which is prepared by a method according to claim 14.

16. Use of a photoprotein according to claim 2, 3, 4 or 15 as a label or a reporter

17. Use of a photoprotein according to claim 2, 3, 4 or 15 as a label or reporter in combination with other reporter genes.

18. A variant of the photoprotein aequorin which exhibits an altered chronological bioluminescence.


SEQUENCE LISTING (7 sequences)

SEQ ID NO: 1 - DNA, 576 bp, artificial sequence
  aequorin mutatnt
atgtcagtcaagcttacaccagacttcgacaacccaaaatggattggacgacacaagcacatgtttaattttcttgatgtcaaccacaatggaaggatctctcttgacgagatggtctacaaggcgtccgatattgttataaacaatcttggagcaacacctgaacaagccaaacgtcacaaagatgctgtagaagccttcttcggaggagctggaatgaaatatggtgtagaaactgaatggcctgaatttatcgaaggatggaaaagactggcttccgaggaattgaaaaggtattcaaaaaaccaaatcacacttattcgtttatggggtgatgcattgttcgatatcattgacaaagaccaaaatggagctatttcactggatgaatggaaagcattcaccaaatctgctggcatcatccaatcgtcagaagattgcgaggaaacattcagagtgtgcgatattgatgaaagtggacagctcgatgttgatgagatgacaagacaacatttaggattttggtacaccatggatcctgcttgcgaaaagctctacggtggagctgtcccctaa

SEQ ID NO: 2 - protein, 196 aa, artificial sequence
  aequorin mutant
MTSEQYSVKLTPDFDNPKWIGRHKHMFNFLDVNHNGRISLDEMVYKASDIVINNLGATPEQAKRHKDAVEAFFGGAGMKYGVETEWPEFIEGWKRLASEELKRYSKNQITLIRLWGDALFDIIDKDQNGAISLDEWKAFTKSDGIIQSSEDCEETFRVCDIDESGQLDVDEMTRQHLGFWYTMDPACEKLYGGAVP

SEQ ID NO: 3 - DNA, 29 bp, artificial sequence
  primer
gaatggcctgaatttatcgaaggatggaa

SEQ ID NO: 4 - DNA, 29 bp, artificial sequence
  primer
ttccatccttcgataaattcaggccattc

SEQ ID NO: 5 - DNA, 28 bp, artificial sequence
  primer
gaatggaaagcattcaccaaatctgctg

SEQ ID NO: 6 - DNA, 28 bp, artificial sequence
  primer
cagcagatttggtgaatgctttccattc

SEQ ID NO: 7 - protein, 196 aa, Aequoria victoria
MTSEQYSVKLTPDFDNPKWIGRHKHMFNFLDVNHNGRISLDEMVYKASDIVINNLGATPEQAKRHKDAVEAFFGGAGMKYGVETEWPEYIEGWKRLASEELKRYSKNQITLIRLWGDALFDIIDKDQNGAISLDEWKAYTKSDGIIQSSEDCEETFRVCDIDESGQLDVDEMTRQHLGFWYTMDPACEKLYGGAVP